(19) United States
(12) Patent Application Publication
(10) Pub. No.: US 2013/0015261 A1
(43) Pub. Date: Jan. 17, 2013

(54) Drag Bag™ (Game lures & cover scent)

(71) Applicants: Jerry Wayne Scarbrough, Bell County, TX (US); Lex A. Mills, Lampasas County, TX (US)

(72) Inventors: Jerry Wayne Scarbrough, Bell County, TX (US); Lex A. Mills, Lampasas County, TX (US)

(21) Appl. No.: 13/135,842

(22) Filed: Jul. 15, 2011

(51) Int. Cl. A61L 9/04

(52) U.S. Cl. 239/34

(57) ABSTRACT

A practically designed, reusable, heated game scent dispenser which allows hunters to choose their preferred liquid or gel game scents for hunting and attracting game animals. It is a durable tri-fold, fabric bag with three compartments made of poly net which allows heated lure to vaporize into the air and onto the ground as it is dragged along with a draw string. The hunter saturates a wick evaporator with his chosen lure and places it into the left wing compartment, folds it over the center compartment where a hand warmer is placed, and then the right wing, with hand warmer, is folded over and secured by the Velcro latches. Drag Bad™ attracts quarry into a position for harvest. It can be hung on a branch where it will continue to attract game. It is especially useful in cold weather, when liquid and gel lures freeze, rendering them ineffective.

### BACKGROUND OF THE INVENTION

### Field of Endeavor

This invention was designed for hunting wild game. It allows hunters to utilize, non-flammable heat to disperse liquid and gel scents and lures currently sold for that purpose. The heat vaporizes the scents or lures as it is dragged along behind the hunter. When the game smells the trail it will follow the hunter because the freshest scent will be laid in his direction of travel. It is most effective in cold and freezing weather when ordinary liquids and gels would be frozen and ineffective.

Other devises currently on the market are drag rags, felt scent pads, candles propane and battery operated heaters. The drag rag is dipped into liquid lure and dragged along the ground or hung in a tree. Freezing temperatures diminish its effectiveness and the scent is depleted as it is dragged on the ground, thus the game it is intended to attract follows it away from the hunter along his back track instead of toward him. The felt dipped pads, candles, propane and battery operated heaters rely on the wind to disburse the scent into the field. Because of the variances of the winds and other elements they are typically limited in effectiveness. Two other inventions which utilize heat are “Buck Hustler” and “Hot Tracks”. “Buck Hustler” requires a portable propane flame heater which vaporizes the scents and disperses them into the air from a fixed location. Its drawbacks are the flames and wind disbursement which is not very effective as discussed above. “Hot Tracks” is not currently on the market, because it requires the lure to be made into a scent impregnated or encapsulated material as determined by the scent manufacturer and utilizes an air activated chemical compound to heat the lure and vaporize it. It must be contained in a vacuum packed container and once opened it chemically heats the impregnated or encapsulated material as determined by the scent manufacturer, and after it is used it must be discarded. It is similar in one respect to the “Drag Bag” and “Drag Rag” in that it can be dragged along the ground. Its major drawback is that the hunter is limited to the manufacturer's desires to make lures and scents available in a material that is some how attached to the air activated chemical heaters. At this time no manufacturer is making such product. The scent and lure candles are open flames and if not carefully watched may start forest fires. In some locals they may be prohibited because of burn bans and dry conditions.

### BRIEF SUMMARY OF THE INVENTION

The “Drag Bag”™ is a reusable tri-fold, heavy duty fabric material which has three compartments in which the hunter places two hand warmers and the desired scented wick. It is folded so the scented wick is sandwiched between the heaters. It has a drag string which attaches to the tongue and tied on the hunter's belt and is dragged behind the hunter as he or she enters their hunting location. It is recommended that the bag touch the ground approximately one foot behind the hunter's foot so as they walk along it bounces on the ground dispersing the lure or scent. It may also be attached to the rear of a motor vehicle and dragged along roads or trails where the hunter intends to hunt. The “Drag Bag”™ is very durable and can use any liquid scent or lure the hunter desires. It can also be hung in a tree or bushes to attract game that smell it in the breeze. It is most desirable because it does not allow the scent or lure to freeze thereby fully utilizing the advantages of the scent or lure to the hunter.

### BRIEF DESCRIPTION OF THE DRAWING

The features and advantages of the “Drag Bag”™ will be fully described in the following specifications The specifications are not meant to restrict the invention to the dimensions and configurations set out, but should be only exemplary and explanatory.

FIG. 1 Full frontal view shows the “Drag Bag”™ heated game scent and lure delivery system, excluding packaging, in its entirety.

### DETAILED DESCRIPTION OF THE INVENTION

Referring to the drawing, and the embodiments of the “Drag Bag”™ invention herein presented by way of illustration, see the “Drag Bag” heated scent game lure system detailed in below

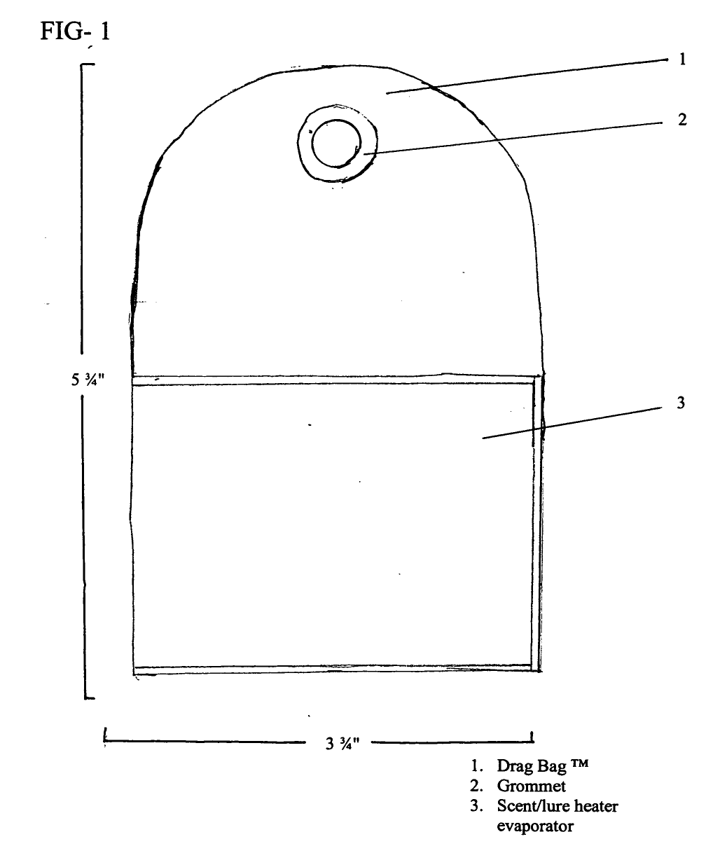

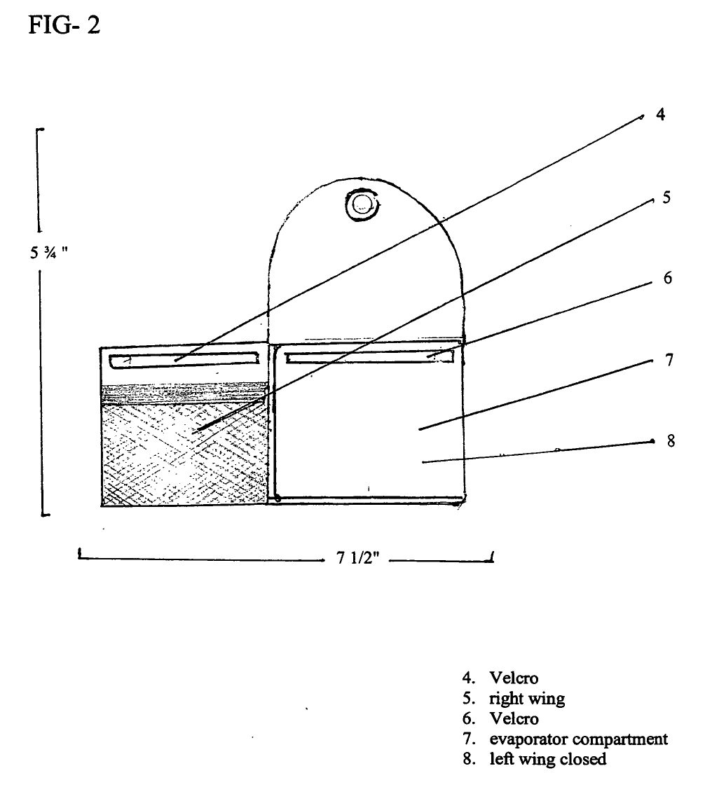

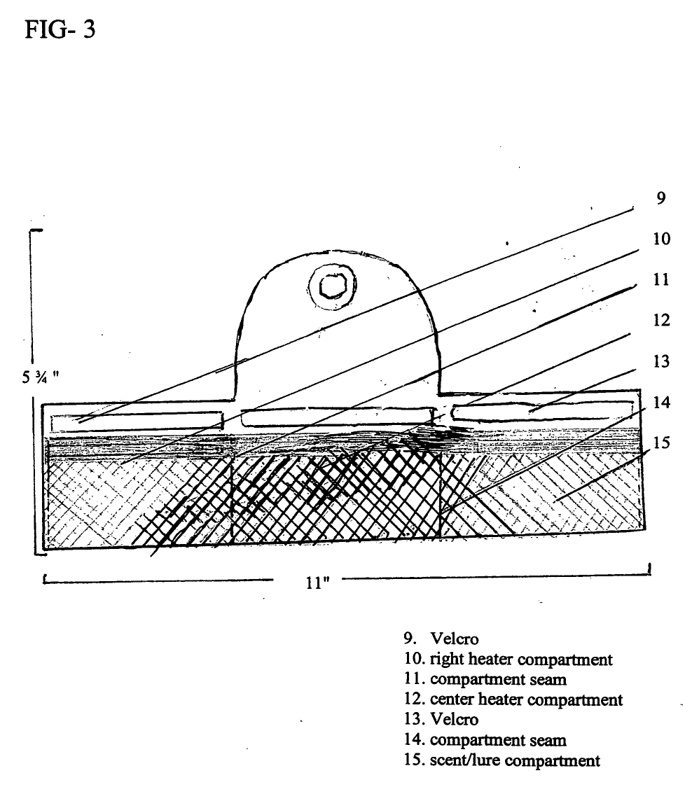

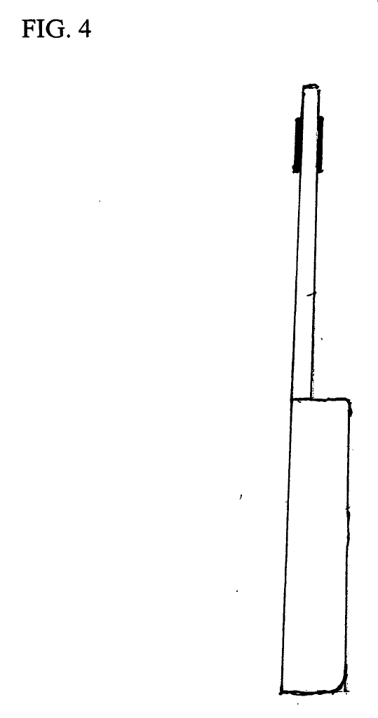

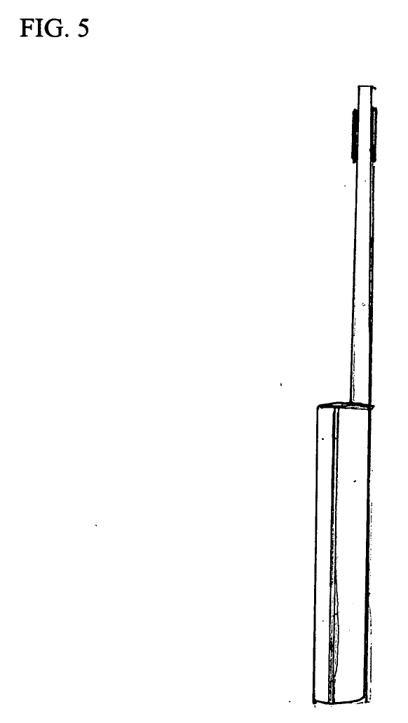

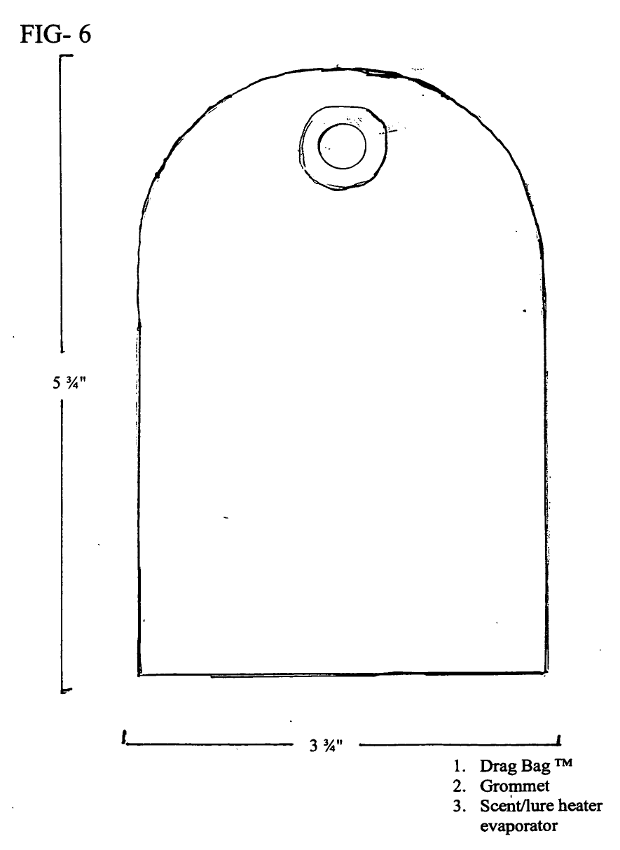

FIG. 1 of the drawing shows the “Drag Bag”™ in its closed ready to use configuration at 1. At the top is a ⅜ inch grommet which is centered 1 inch from the top of the tongue at 2. It is made of a very durable fabric material with three compartments which fold into a pouch or bag where the scent and heaters are placed and folded into a sandwich configuration with the scent or lure in the middle surrounded by the hand warmers, or a single hand warmer can be sandwiched between two scent pads, at 3.

FIG. 2 of the drawing illustrate the “Drag Bag”™ with the right wing heater compartment open, at 5. The left wing of the “Drag Bag”™ in which the scent or lure is placed is closed with its fabric backing exposed at 7. When it is closed it is secured by the Velcro latched at 4 & 6 forming a compartment at 8.

FIG. 3 of the drawing shows the “Drag Bag”™ in its fully open configuration with both wings out for loading the hand warmers at 10 & 12 and scent or lure at 15, or vice versa. Note the compartments are made with a durable nylon mesh or netting material which forms a pocket along the bottom of the bag which is divided into three equal sizes by seams at 11 & 14. When the left wing is folded over the center compartment they are firmly attached by the Velcro stripes at 9 & 13. This forces the evaporated scent to escape from the bottom as it is dragged behind the hunter, or hung in a tree.

FIG. 4. Of the drawing illustrates the right edge of the “Drag Bag”™ when it is closed and ready for use.

FIG. 5 of the drawing illustrates the left edge of the “Drag Bag”™ when it is folded ready for use FIG. 6 illustrates the back side of the “Drag Bag” ™ when it is folded up and ready for use. The dimensions of the “Drag Bag”™ in FIG. 1 are 5¾ by 3¾ inches. Commercially available had warmers sold in sporting goods stores and other outlets fit nicely into the compartments. Small absorbent tampons are used to absorb the scent from their containers and using the string on the pads they drop nicely into the scent or lure compartment without any spills or drips. They can be reused by just pouring the scent over them in place in the “Drag Bag”™.

What we claim our invention is:

1. A durable, compact, reusable heat activated game scent or lure dispensing device which utilizes commercially available liquids and gels widely distributed and sold in the sporting goods market.

2. The durable, compact, reusable, heat activated game scent or lure dispensing device of claim 1, wherein heat is used to activate and evaporate the game scents or, lures is safe for use in all locations and especially effective in cold or freezing temperatures where other liquid and gel dispensers freeze, rendering them useless.

3. The durable, compact, reusable, heat activated game scent or lure dispensing device of claim 1, wherein the tri-fold design allows the heaters to directly contact the scent or lure pad and increases the evaporation and distribution of the scent.

4. The durable, compact, reusable, heat activated game scent or lure dispensing device of claim 1, wherein its design allows it to be hung in a tree or on bushes to lure game by using the wind or breezes.

5. The durable, compact, reusable, heat activated game scent or lure dispensing device of claim 1, wherein it may be dragged by hunter, or other means such as motor vehicles, or RVs.

6. The durable, compact, reusable, heat activated game scent or lure dispensing device of claim 1, wherein it is more effective because the vaporized scents and lures it dispenses are stronger in the direction of the hunter, and when the desired game follows the lure the strongest lure scent will be closer to the hunter, not his back trail.

7. The durable, compact, reusable, heat activated game scent or lure dispensing device of claim 1, wherein it can be used with a single heating pad and two scent lure pads for stronger disbursement in warmer temperatures.
